(19) United States
(12) Patent Application Publication
(10) Pub. No.: US 2007/0213702 A1
(43) Pub. Date: Sep. 13, 2007

(54) Medical procedure carried out via a natural opening

(75) Inventors: Takahiro Kogasaka, Tokyo (JP); Manabu Miyamoto, Tokyo (JP); Saori Takeuchi, Tokyo (JP); Kiyotaka Matsuno, Tokyo (JP); Takumi Dejima, Tokyo (JP); Ken Yamatani, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/371,565

(22) Filed: Mar. 8, 2006

(51) Int. Cl. A61B 18/18

(52) U.S. Cl. 606/32; 606/47

(57) ABSTRACT

A medical procedure carried out via a natural opening, comprising: fastening the distal end of thread members to a tissue in a body cavity in proximity to a targeted incision site via a first introduction member that is inserted into a body cavity through a natural opening of an examination subject; inserting a second introduction member having a cutting instrument attached to the distal end thereof into a body cavity through a natural opening of an examination subject and disposing the cutting instrument in proximity to the targeted incision site; and cutting the tissue by pressing the cutting instrument against the tissue while pulling the proximal end sides of the thread members.

### BACKGROUND OF THE INVENTION

1. Field of the Invention

The present invention relates to medical procedure carried out via a natural opening.

2. Description of Related Art

When carrying out medical procedures that include the observation and the treatment of the internal organs of the human body, laparotic surgery is known in which, instead of forming a large incision in the abdominal wall, a plurality of openings are formed in the abdominal wall, instruments such as a laparoscope and forceps are inserted into respective openings, and the manipulation is carried out. In this type of surgery, because only forming small openings in the abdominal wall is necessary, there is the advantage that the burden on the patient becomes small.

Over the past few years, a method for further reducing the burden on patients has been proposed wherein a manipulation is carried out by inserting a flexible endoscope through a natural opening of the patient, such as the mouth, nose, or rectum. An example of such a manipulation is disclosed in U.S. Pat. No. 5,458,131.

In this method, a flexible endoscope is inserted through the patient's mouth, an incision is formed in the stomach wall, and the distal portion of the endoscope is passed into the abdominal cavity through this opening. In addition, while using the endoscope as an apparatus to observe the inside of the abdominal cavity, the desired manipulation is carried out in the abdominal cavity by using instruments that are passed through the endoscope or instruments that are inserted through another opening.

### SUMMARY OF THE INVENTION

An object of the present invention is to provide one method wherein the cutting of a targeted incision site of a tissue in a body cavity can be carried out more accurately during a medical procedure carried out via a natural opening.

A medical procedure carried out via a natural opening according to an aspect of the present invention includes fastening the distal end of thread members to a tissue in a body cavity in proximity to a targeted incision site via a first introduction member that is inserted into a body cavity through a natural opening of an examination subject, inserting a second introduction member having a cutting instrument attached to the distal end thereof into a body cavity through a natural opening of an examination subject and disposing the cutting instrument in proximity to the targeted incision site, and cutting the tissue by pressing the cutting instrument against the tissue while pulling the proximal end sides of the thread members.

### BRIEF DESCRIPTION OF THE DRAWINGS

FIG. 1 is a schematic drawing showing the overall overtube that is used in a medical procedure according to a first embodiment.

FIG. 2 is a drawing showing the essential components of the overtube that is used in a medical procedure according to a first embodiment.

FIG. 3 is a perspective view showing the distal end portion of the overtube that is used in a medical procedure according to a first embodiment.

FIG. 4A is a cross-sectional view along III-III in FIG. 3.

FIG. 4B is a cross-sectional view along IV-IV in FIG. 4.

FIG. 5 is a partial enlarged cross-sectional view showing the electrode control portion of the overtube that is used in a medical procedure according to a first embodiment.

FIG. 6 is a cross-sectional view showing the state in which the anchors are installed in the puncture needles of the overtube that is used in a medical procedure according to a first embodiment.

FIG. 7 is a partial cross-sectional view showing the needle control portion of the overtube that is used in a medical procedure according to a first embodiment.

FIG. 8 is a cross-sectional view in proximity to the endoscope lock button of the overtube that is used in a medical procedure according to a first embodiment.

FIG. 9 is a schematic drawing of the overall endoscope showing an example of the device that is passed through the overtube that is used in a medical procedure according to a first embodiment.

FIG. 10 is a flowchart of a medical procedure according to a first embodiment.

FIG. 11 is a drawing for explaining the state in which the endoscope has been inserted in the overtube in a medical procedure according to a first embodiment.

FIG. 12 is a drawing for explaining the state in which the overtube has been introduced to a targeted incision site in a medical procedure according to a first embodiment.

FIG. 13 is a drawing for explaining the state in which a portion of the stomach wall is sucked into the overtube in a medical procedure according to a first embodiment.

FIG. 14 is a drawing for explaining the state in which the stomach wall that has suction applied has been punctured by an injection needle in a medical procedure according to a first embodiment.

FIG. 15 is a drawing for explaining the state in which the stomach wall that has suction applied has been punctured by the puncture needles of the overtube while the abdomen is insufflated with air that has been delivered from the injection needle in a medical procedure according to a first embodiment.

FIG. 16 is a drawing for explaining the state in which the anchors have been released from the puncture needles in a medical procedure according to a first embodiment.

FIG. 17 is a drawing for explaining the state in which the stomach wall has been cut by the cutting electrode of the overtube while pulling the proximal end sides of the thread members in a medical procedure according to a first embodiment.

FIG. 18 is a drawing showing FIG. 17 after being rotated 90 degrees.

FIG. 19 is a drawing for explaining the state in which the endoscope has been inserted into the abdominal cavity in a medical procedure according to a first embodiment.

FIG. 20A is a drawing for explaining a procedure for suturing the opening portion that has been cut by using two retained anchors and the thread members extending therefrom in a medical procedure according to a first embodiment.

FIG. 20B is a drawing for explaining a procedure for suturing the opening portion that has been cut by using two retained anchors and the thread members extending therefrom in a medical procedure according to a first embodiment.

FIG. 21 is a cross-sectional view showing a modified example of the distal end portion of an overtube that is used in a medical procedure according to a first embodiment.

FIG. 22 is a drawing for explaining the state before the stomach wall has been cut by the cutting instrument while pulling the proximal end sides of the thread members in a medical procedure according to a second embodiment.

FIG. 23 is a drawing for explaining the state after the stomach wall has been cut by the cutting instrument while pulling the proximal end sides of the thread members in a medical procedure according to a second embodiment.

FIG. 24 is a perspective view of the cutting instrument that is used in a medical procedure according to a second embodiment.

FIG. 25 is a perspective view of another cutting instrument that is used in a medical procedure according to a second embodiment.

FIG. 26 is a perspective view of the tip of the cutting instrument that is used in a medical procedure according to a second embodiment.

FIG. 27A is a side view of the cutting instrument shown in FIG. 26.

FIG. 27B is a frontal view of the cutting instrument shown in FIG. 26.

FIG. 28A is a side view of another tip of a cutting instrument that is used in a medical procedure according to a second embodiment.

FIG. 28B is a frontal view of the tip of a cutting instrument that is shown in FIG. 28A.

FIG. 29 is a perspective view of a cutting instrument that is used in a medical procedure according to a third embodiment.

FIG. 30A is a drawing for explaining the state before the stomach wall is cut by the cutting instrument while pulling the proximal end sides of the thread members in a medical procedure according to a third embodiment.

FIG. 30B is a drawing for explaining the state after the stomach wall has been cut by the cutting instrument while pulling the proximal end sides of the thread members in a medical procedure according to a third embodiment.

FIG. 31 is a perspective view of another cutting instrument that is used in a medical procedure according to a third embodiment. FIG. 32A is a drawing for explaining the state before the stomach wall is cut by another cutting instrument while pulling the proximal end sides of the thread members in a medical procedure according to a third embodiment.

FIG. 32B is a drawing for explaining the state after the stomach wall has been cut by another cutting instrument while pulling the proximal end sides of the thread members in a medical procedure according to a third embodiment.

FIG. 33 is a perspective view of an endoscope that is used in a medical procedure according to a fourth embodiment.

FIG. 34 is a drawing for explaining the state before the stomach wall is cut by a cutting instrument while pulling the proximal end sides of the thread members in a medical procedure according to a fourth embodiment.

FIG. 35 is a drawing for explaining the state after the stomach wall has been cut by another cutting instrument while pulling the proximal end sides of the thread members in a medical procedure according to a fourth embodiment.

### DETAILED DESCRIPTION OF THE INVENTION

The embodiments are explained in detail below. Note that below, identical structural elements are denoted by identical reference numerals, and redundant explanations thereof are omitted.

### First Embodiment

The overtube 1 according to the present embodiment provides a device insertion portion which is inserted into the body of an examination subject, and is used as a guide tube when inserting into the body a device such as an endoscope (in order to simplify the explanation, in the following explanation this may simply referred to as a “device” or an “endoscope”) for performing medical procedures in the body.

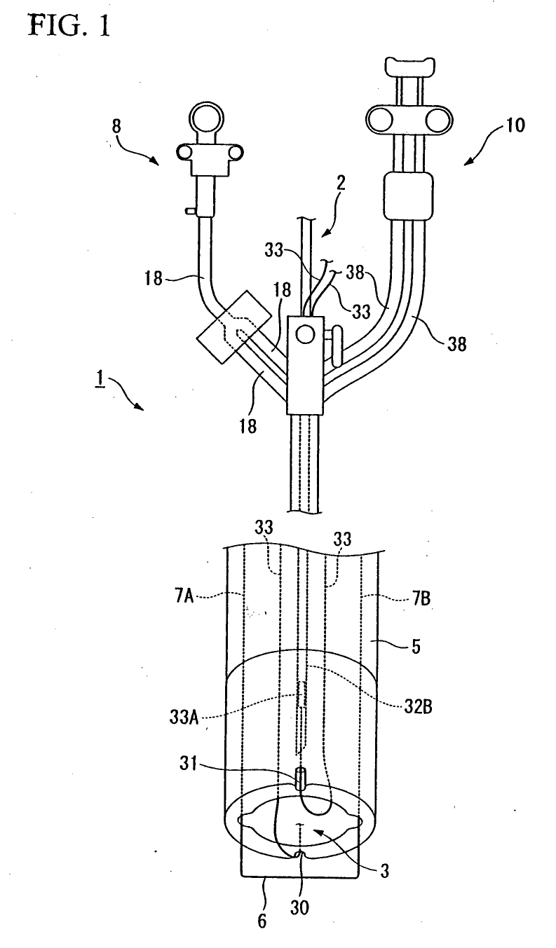

As shown in FIG. 1, this overtube 1 has an insertion portion 5 that has a first lumen 3 having a distal end that opens in the direction of insertion into the examination subject and into which an endoscope 2, which is an example of a device that extends in an axial direction, is inserted so as freely advance and retract, and is inserted into a luminal organ such as the stomach or the abdominal cavity of a patient; a cutting electrode (tissue cutting portion) 6 that traverses the distal end side of the first lumen 3 and, in the traversing state, is releasably disposed in the insertion portion 5, and cuts the body tissue of the patient; electrode controlling wires (controlling members) 7A and 7B that are each connected to the cutting electrode and disposed so as to freely advance and retract in the insertion portion 5; an electrode controlling portion (operating portion) 8 that advances or retracts the electrode controlling wires 7A and 7B in the first lumen 3; and a needle control portion 10 for controlling the puncture needles 32A and 32B and the pushers 35 described below. Note that in this embodiment, the cutting electrode 6 and the electrode controlling wires 7A and 7B are formed by one wire. However, the cutting electrode 6 and the electrode controlling wires 7A and 7B may be formed by separate members and connected together. In addition, the cutting electrode 6 may be provided so as not to advance or retract with respect to the insertion portion 5.

Note that in the first embodiment, the overtube 1 forms first and second introduction members in which the distal ends of the thread members 33 are inserted into a body cavity of the patient and a cutting electrode 6, which is a cutting instrument, is introduced into a body cavity.

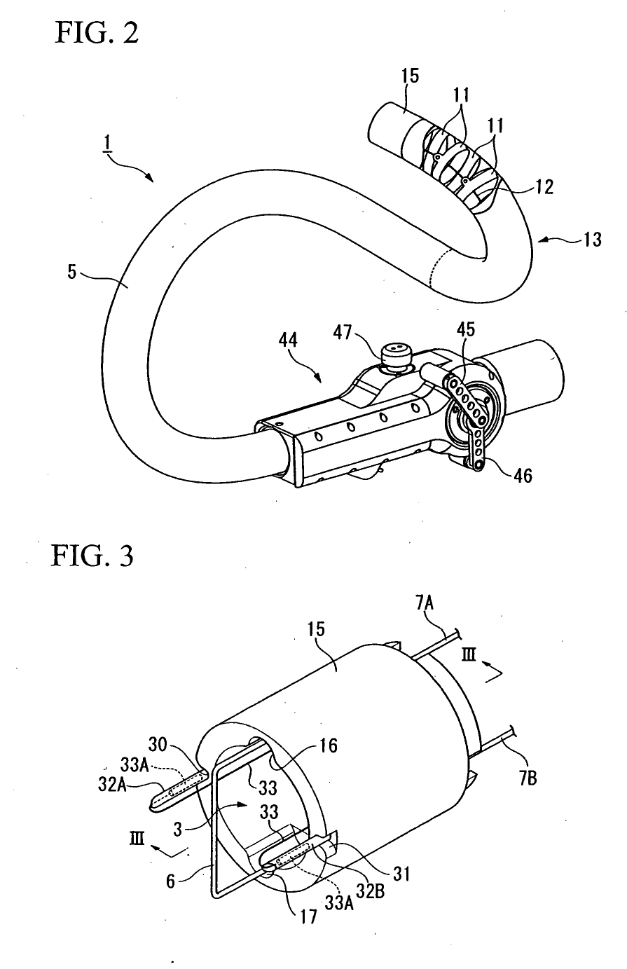

As shown in FIG. 2, the insertion portion 5 is elongated and flexible, and like a typical flexible endoscope, a bending portion 13 is provided at the distal end side of the insertion portion 5. In the bending portion 13, a plurality of segmented rings 11 is connected along the bending wires 12. Here, a bending portion 13 that is actively bent by the operation of the operator is not provided. Rather, the insertion portion may be formed in the shape of a flexible tube and passively bent by conforming to the bent shape of a device such as an endoscope. As shown in FIG. 3, a short tubular distal end portion 15 is provided farther toward the distal end than the bending portion 13.

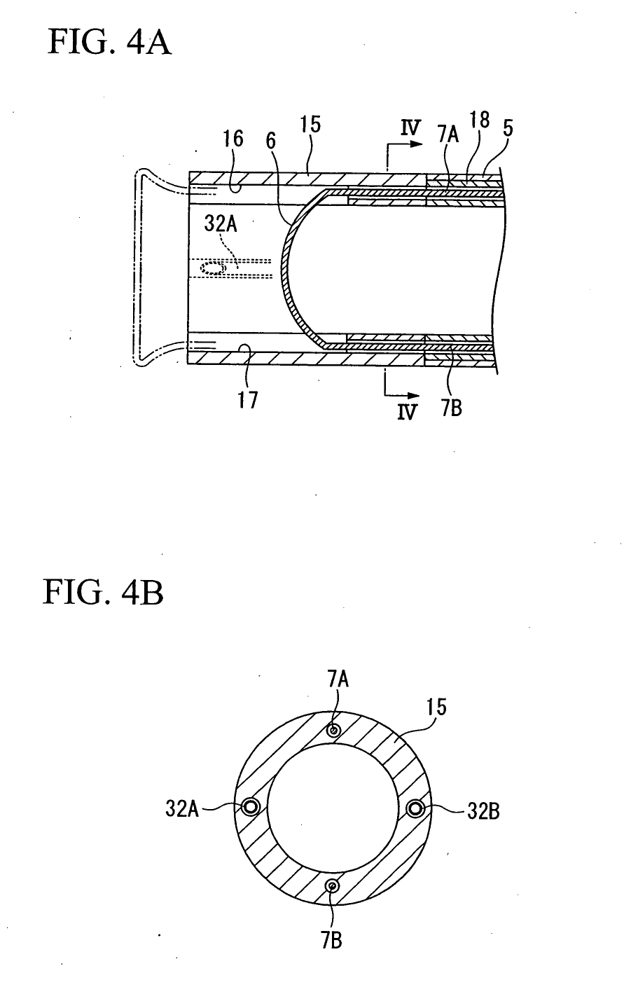

The cutting electrode 6 is, for example, a stainless steal wire that is capable of high frequency conduction. The cutting electrode 6 is disposed so as to traverse the center portion of the lumen 3 in a direction that is perpendicular to the axial direction of the insertion portion 5. Specifically, as shown in FIG. 3 and FIG. 4, one end side of the cutting electrode 6 is connected to an electrode controlling wire 7A that is passed through the first inner groove 16 formed on the outer edge of the wall that defines the lumen 3. The lumen 3 is defined by the inner surface of the distal end portion 15. The other end side of the cutting electrode 6 is connected to an electrode controlling wire 7B that is passed through a second inner groove 17 formed in the outer edge of the wall that defines the lumen 3 at a substantially symmetrical position with respect to the first inner groove 16 across the center of the first lumen 3. The length of the cutting electrode 6 is formed so as to be longer than the inner diameter of the lumen 3, and as shown in FIG. 4, the cutting electrode 6 is accommodated in a bent state inside the lumen 3 and can move along the first inner groove 16 and the second inner groove 17. Note that in the embodiment shown in FIG. 4, the length of the cutting electrode 6 has been set so as to be longer than the inner diameter of the lumen 3, but this is not limiting. The length may be set such that the cutting electrode 6 is accommodated in the lumen 3 in a state that is not bent (for example, a length substantially identical to the inner diameter of the lumen 3).

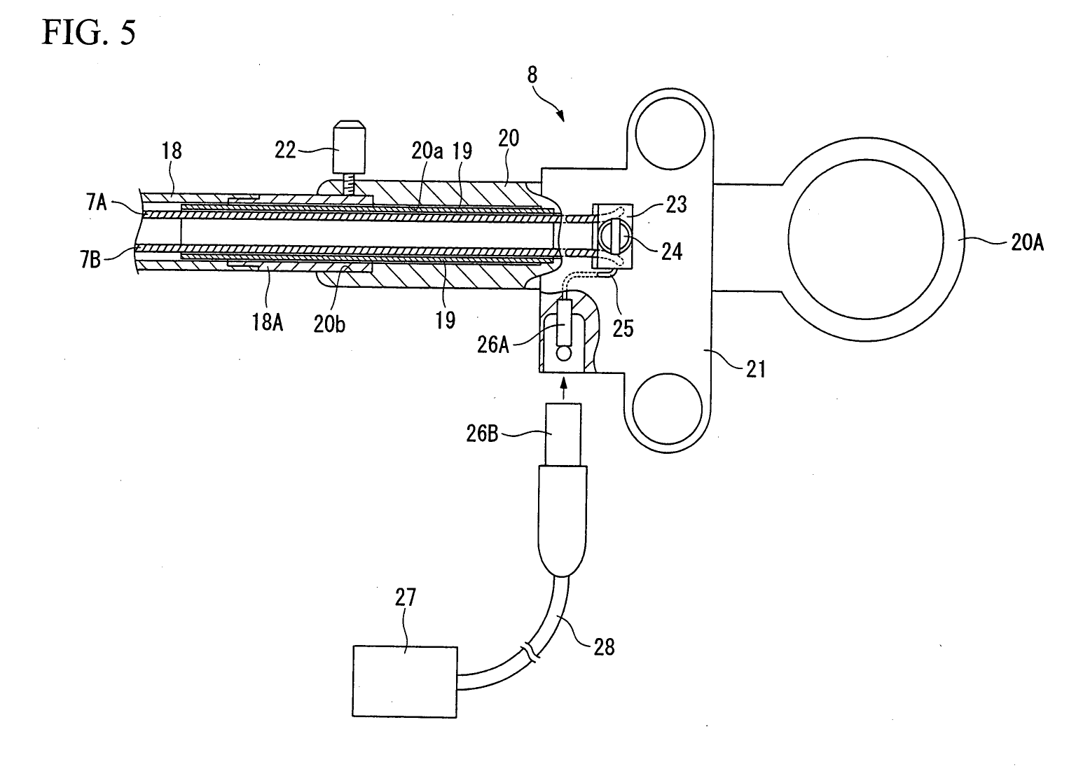

The electrode controlling wires 7A and 7B are passed through the electrode tube 18. There is one electrode tube 18 at the proximal end side that projects from the insertion portion 5, but in order to accommodate the electrode controlling wires 7A and 7B separately in the insertion portion 5, as shown in FIG. 1, the two wires are separated along the way and passed through the insertion portion 5, and the distal ends thereof are connected to the distal end portion 15. As shown in FIG. 5, the proximal ends of the electrode controlling wires 7A and 7B pass through one rigid operating pipe 19 that is disposed so as to project from the distal end of a control handle 21 described below.

The electrode controlling portion 8 has a control body portion 20 that is connected to the proximal end of an electrode tube 18 and a control handle 21 that is disposed on the control body portion 20 so as to freely advance and retract. A through hole 20a is provided through which the electrode controlling wires 7A and 7B and the operating pipe 19 are inserted. An engaging hole 20b that engages the rigid portion 18A, which is disposed at the proximal end of the electrode tube 18, is formed on the distal end of the controlling body portion 20, and the electrode tube 18 is fastened to the engaging hole 20b by a bis 22. A grasping portion 20A is disposed at the proximal end of the control body portion 20.

A connecting plate 23 is disposed on the control handle 21, and is electrically connected to the end portion of the electrode controlling wires 7A and 7B that have been passed through the operating pipe 19. A fastening screw 24 is disposed on the connecting plate 23, and by clamping the fastening screw 24 onto the connecting plate, the electrode controlling wires 7A and 7B are fastened and electrically connected. The connecting plate 23 is electrically connected to the connecting terminal 26A that is disposed on the control handle 21 via the electric wiring 25. A connecting terminal 26B that is disposed on the distal end of a power cord 28, which extends from the high frequency power source 27, is fastened to and released from the connecting terminal 26A. A finger catch portion 21A is also disposed on the control handle 21.

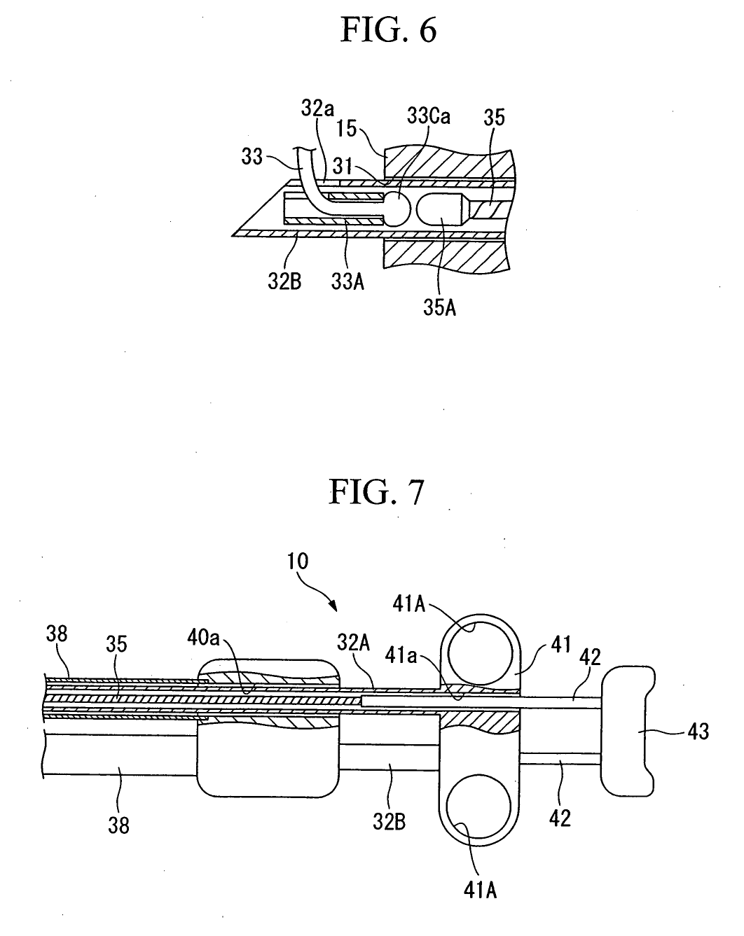

On the outer surface of the distal end portion 15 of the overtube 1, a first outer groove 30 and a second outer groove 31 are formed at a position along the distal end portion 15 toward the distal end at positions perpendicular to the direction that joins the first inner groove 16 and the second inner groove 17. Two puncture needles (hollow needles) 32A and 32B that advance and retract along the lumen 3 are each disposed so as to be able to advance and retract in the first outer groove 30 and the second outer groove 31. As shown in FIG. 1 and FIG. 6, anchors 33A that are attached to the distal ends of the thread members 33 are held in each of the puncture needles 32A and 32B.

An anchor 33A has a cylindrical shape, and a slit 33Aa having a length approximately half that of the overall length thereof is formed at one end side along the longitudinal direction. A thread member 33 is inserted from the slit 33Aa and is passed through the inside of an anchor 33A in the longitudinal direction thereof after being bent at some point. A large diameter portion 33Ca having a diameter that is larger than the inner diameter of the anchor 33A is formed on the distal end of a thread member 33, and thereby, the thread member 33 cannot be extracted from the anchor 33A. As shown in FIG. 1 and FIG. 3, the proximal end sides of the thread members 33 bend back after reaching the edge of the distal end portion 15 of the overtube 1, pass through the lumen 3 of the overtube 1, and are exposed to the outside from the proximal end side opening of the overtube 1. Note that as necessary a disk having a diameter that is larger than the inner diameter of the overtube 1 may be attached or a rod member having a length that is longer than the inner diameter of the overtube 1 may be attached to the proximal end portion of the thread members 33 in order to prevent the proximal end sides of the thread members 33 from entering into the overtube 1. The disk or the rod portions act as an engaging portion, and thereby the proximal end portions of the thread members 33 are prevented from being unintentionally drawn into the overtube 1. In addition, it is possible to grasp the engaging portion when the proximal end portions of the thread members 33 are pulled.

Note that the thread members 33 and the anchors 33A are used during the suturing step described below. This will be explained in detail below.

A pusher 35 is disposed so as to freely advance and retract inside each of the puncture needles 32A and 32B. The puncture needles 32A and 32B and the pushers 35 are respectively accommodated in the two outside sheaths 38. The two outside sheaths 38 are each passed through the insertion portion 5, and the distal ends thereof are connected to the distal end portion 15. A rigid pushing member 35A is disposed on the distal end of a pusher 35. As shown in FIG. 6, a slit 32a, through which a thread member 33 is passed, is formed on the distal end of the puncture needles 32A and 32B.

As shown in FIG. 1 and FIG. 7, the needle controlling portion 10 includes a sheath grasping portion 40 that is disposed so as to connect to the proximal ends of the two outside sheaths 38; a needle controlling handle 41 that is disposed so as to connect to the proximal ends of the two puncture needles 32A and 32B, which pass through the through hole 40a provided in the sheath grasping portion 40 so as to freely advance and retract; and a pusher connecting portion 43 that connects together the end portions of the rod-shaped rigid portions 42 that are connected to the proximal ends of the two pushers 35 that pass through the through holes 41a provided on the needle controlling handle 41 so as to freely advance and retract. A finger catch 41A is disposed on the needle controlling handle 41. Note that the needle controlling handle 41 and the pusher connecting portion 43 may be divided into two positions, such that the two puncture needles 32A and 32B and the two pushers 35 can each be controlled independently.

As shown in FIG. 2, a distal handle 44 having a diameter that is larger than that of the insertion portion 5 is disposed at the proximal end of the insertion portion 5 of the overtube 1. A bending lever 45 that is connected to the proximal end side of the bending wire 12 for carrying out bending control of the bending portion 13; a bending lock lever 46 that fastens the position of the bending lever 45 at an arbitrary position; and an endoscope lock button 47 for fastening the endoscope 2 with respect to the lumen 3 when the endoscope 2 is passed through the lumen 3 are disposed on the distal handle 44.

The distal end sides of the bending wires 12 are fastened to the distal end portion 15, and in this embodiment, two bending wires 12 pass through the inside of the insertion portion 5, and the distal ends thereof are fastened at substantially opposing positions of the distal end portion 15 across the center of the first lumen 3. Note that in this embodiment, two bending wires 12 are provided and the bending portion 13 is formed so as to bend in two directions. However, this is not limiting, and like the bending portions of well-known endoscopes, a structure may be used wherein four bending wires 12 and two bending control levers 45 are provided, and the bending portion bends in four dictions.

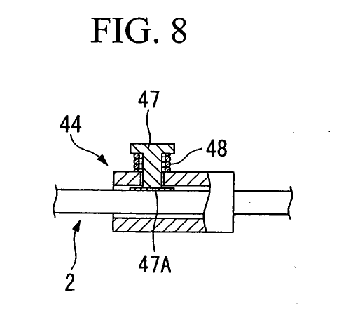

As shown in FIG. 8, a broad pushing portion 47A is disposed at the distal end of the endoscope lock button 47, and during normal usage, the endoscope lock button 47 is urged outward by the spring 48 in the radial direction of the distal handle 44. When it is necessary to insert and fasten the endoscope 2 inside portion 5, the endoscope lock button 47 is pressed inward in the radial direction, and thereby the pushing portion 47A presses the endoscope 2 so that the endoscope 2 is relatively fastened due to friction. Note that alternatively the endoscope lock button 47 may be pressed to release the friction.

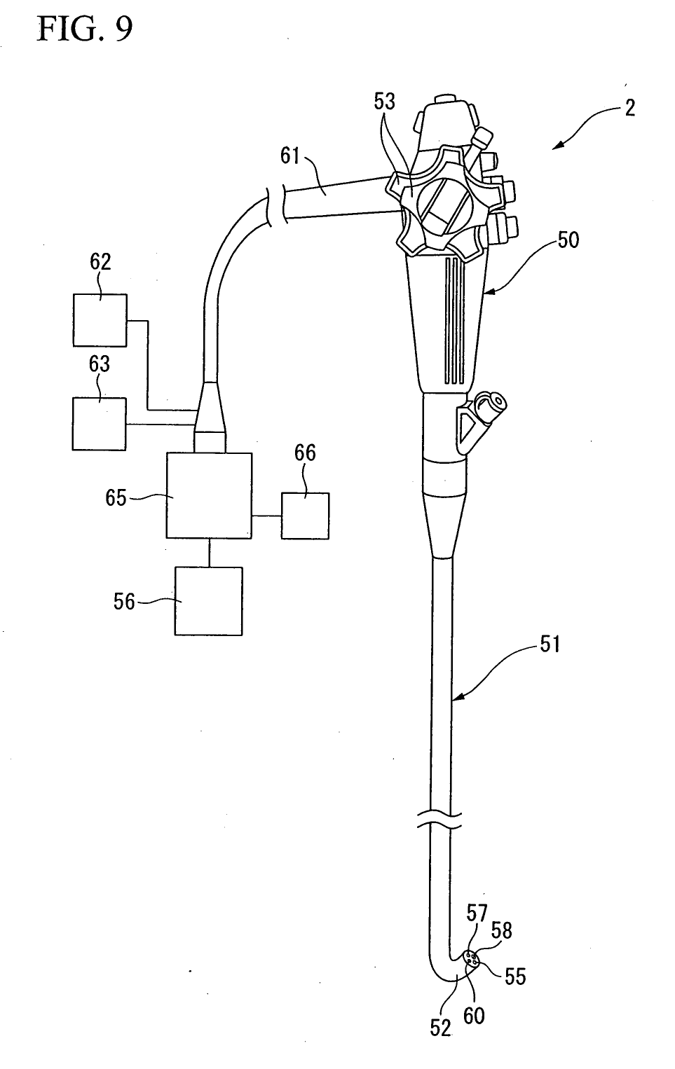

The endoscope 2 that is inserted into this overtube 1 is, as for example, as shown in FIG. 9, a flexible endoscope, and the elongated and flexible endoscope insertion portion 51 that is inserted into the patients body extends from the endoscope controlling portion 50 that is controlled by the technician. The endoscope distal end portion 52 of the endoscope insertion portion 51 can be bent by controlling the angle knob 53 that is disposed on the endoscope controlling portion 50. An objective lens 55, a distal end surface of an optical fiber 57 that guides light from a light source apparatus 56 and is disposed outside the body, and the distal end openings of the channels 58 and 60 are disposed at the endoscope distal end portion 52. The channel 58 is a passage that is connected to the air and water feeding apparatus 62 and the suction apparatus 63, which are disposed outside the body via the universal cable 61, and used for supplying fluids to and discharging fluids from the inside of the body. In addition, the channel 60 is a passage for inserting and removing instruments, and is disposed between the 6 o'clock and 8 o'clock positions of the endoscope insertion portion 51. Note that, for example, two channels for instruments may be provided, and the number of channels is not limited to one. The observed images input by the objective lens 55 are displayed on a monitor 66 via a control portion 65.

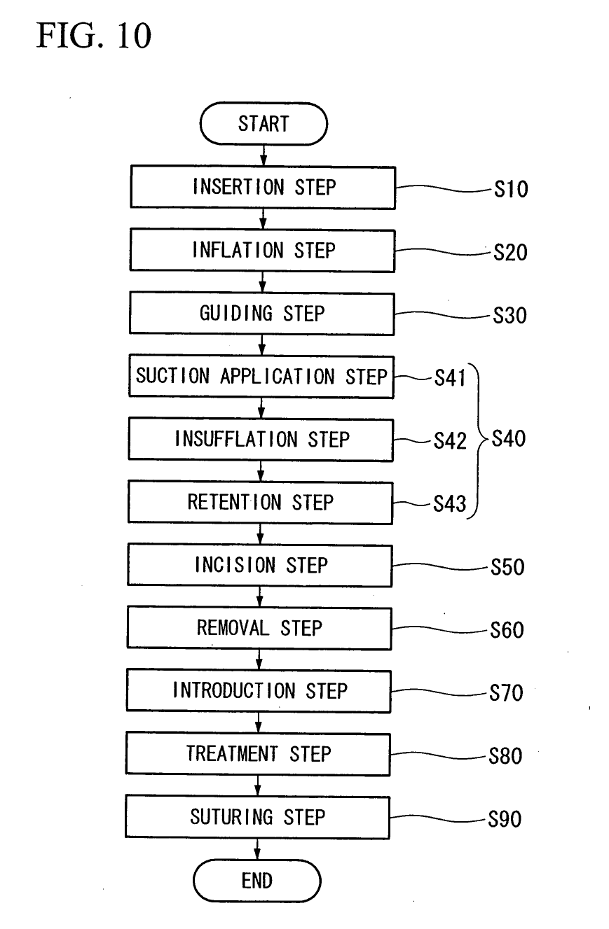

Next, the operation of the present embodiment will be explained along with a medical procedure carried out via a natural opening shown in the flowchart in FIG. 10 using the overtube 1. Note that in the following, the targeted incision site T is on the anterior wall of the stomach ST, and a manipulation is explained in which a medical procedure is carried by inserting the endoscope 2 through the mouth M of the patient PT into the stomach (a luminal organ) ST and then inserting the insertion portion 5 of the endoscope 2 into the abdominal cavity AC through an opening the stomach wall. In addition, in the embodiment explained below, the endoscope 2 is inserted as a device through the mouth M of the patient PT into the body, and the abdominal cavity AC is approached by forming an opening SO in the anterior wall of stomach ST. However, the natural opening into which the endoscope 2 is inserted is not limited to the mouth M, and the natural opening may be the rectum, nose, or the like. Furthermore, preferably the opening SO is formed in the anterior wall of the stomach ST. However, this is not limiting, and an opening in the wall of a luminal organ or vessel into which the device may be inserted as a natural opening may be formed in another part of the stomach ST, the esophagus, the small intestine, the large intestine or the like.

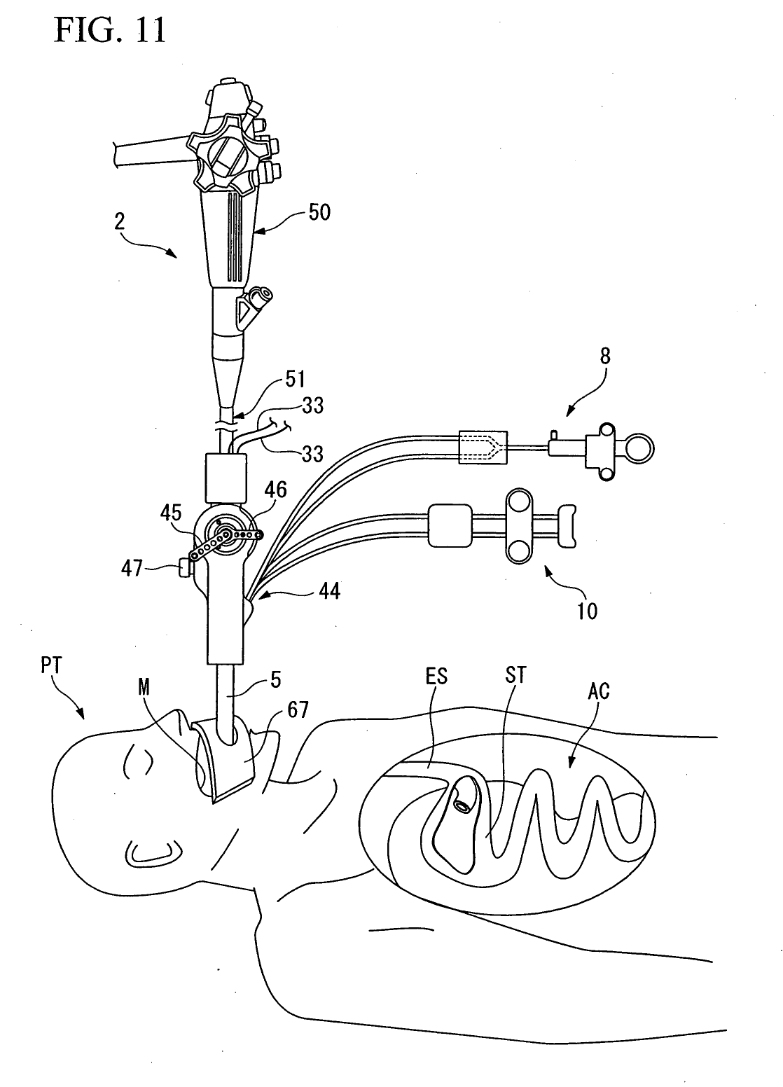

First, while the patient PT is lying down facing upward, an insertion step (S10) is carried out in which the endoscope 2 is passed through the lumen 3 formed in the insertion portion 5 of the overtube 1, and the insertion portion 5 of the overtube 1 and the endoscope 2 are inserted into the stomach (a luminal organ) ST through the mouth M of the patient PT while observing the inside of the body cavity using the endoscopic images. As shown in FIG. 11, a mouthpiece 67 is mounted in the mouth of the patient PT, and with the endoscope 2 passed through the lumen 3, the overtube 1 and the endoscope 2 are inserted through the mouthpiece 67 into the esophagus ES. Note that both the cutting electrode 6 and the puncture needles 32A and 32B are disposed at an initial position, where they are accommodated in the distal end portion 15.

Next, as a inflation step (S20), air is supplied to the stomach ST via the channel 58 of the insertion portion 5 from the air and water feeding apparatus 62 to distend the stomach ST.

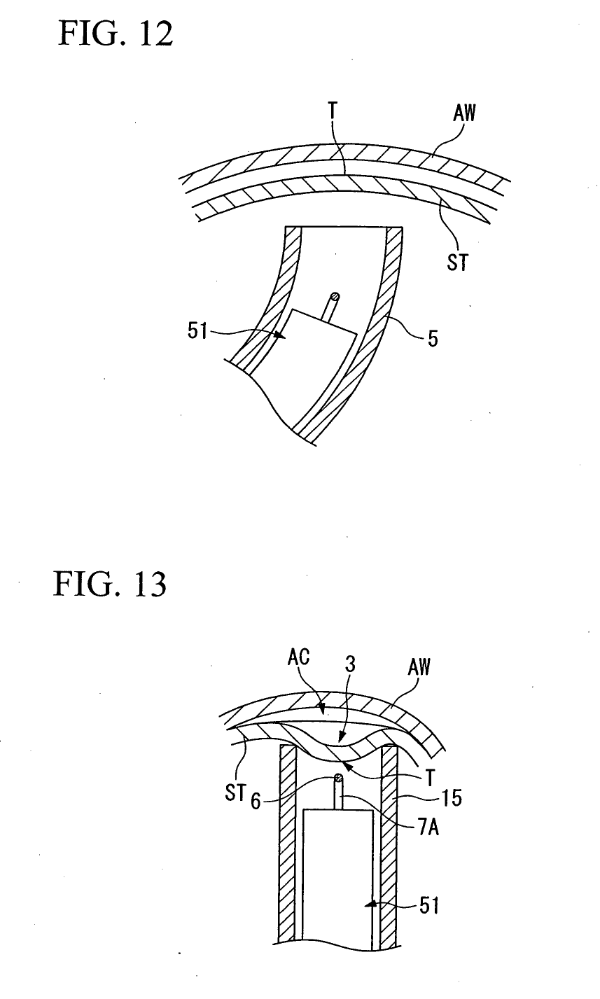

Next, while confirming the targeted incision site T by using the endoscope 2, which also serves as an observation device, the processes proceeds to a guidance step (S30) in which the insertion portion 5 of the overtube 1 is guided up to the targeted incision site T. First, after the endoscope insertion portion 51 of the endoscope 2 has been inserted into the stomach, while observing the interior of the stomach ST via the observing apparatus 55 disposed in the endoscope insertion portion 51, the distal end of the endoscope insertion portion 51 is brought up to the targeted incision site T by operating the angle knob 53. Next, while identifying the targeted incision site T, using the endoscope insertion portion 51 as a guide, the insertion portion 5 of the overtube 1 is pressed, and as shown in FIG. 12, the distal end 15 of the overtube 1 approaches in proximity to the targeted incision site T.

Next, the process proceeds to a needle moving step (S40) in which the puncture needles 32A and 32B, which are disposed at the distal end side of the insertion portion 5, are advanced and retracted along the first lumen 3. First, as a suction application step (S41), while the distal end portion 15 is abutted against the stomach wall so as to include the targeted incision site T, a suction is applied to the stomach wall by the suction apparatus 63 via the channel 58. At this time, as shown in FIG. 13, a portion of the stomach wall is sucked into the first lumen 3 of the distal end portion 15 from the distal end opening 3A. Thereby, a space is established between the outer stomach wall and the abdominal cavity AC. Here, as a device for applying suction to the stomach wall, a method in which the channel 58 of the endoscope 2 is used is not limiting. For example, a space formed between the inner surface of the first lumen 3 of the overtube 1 and the outer circumference of the insertion portion of a device such as an endoscope 2 that has been inserted into the first lumen 3 may be used as a suction passage, and a suction may be established by being attached to a suction apparatus 63. In this case, the suction effect may be further improved by having a valve (not illustrated) in the established space to inhibit the flow of fluids between the inside and outside of the body.

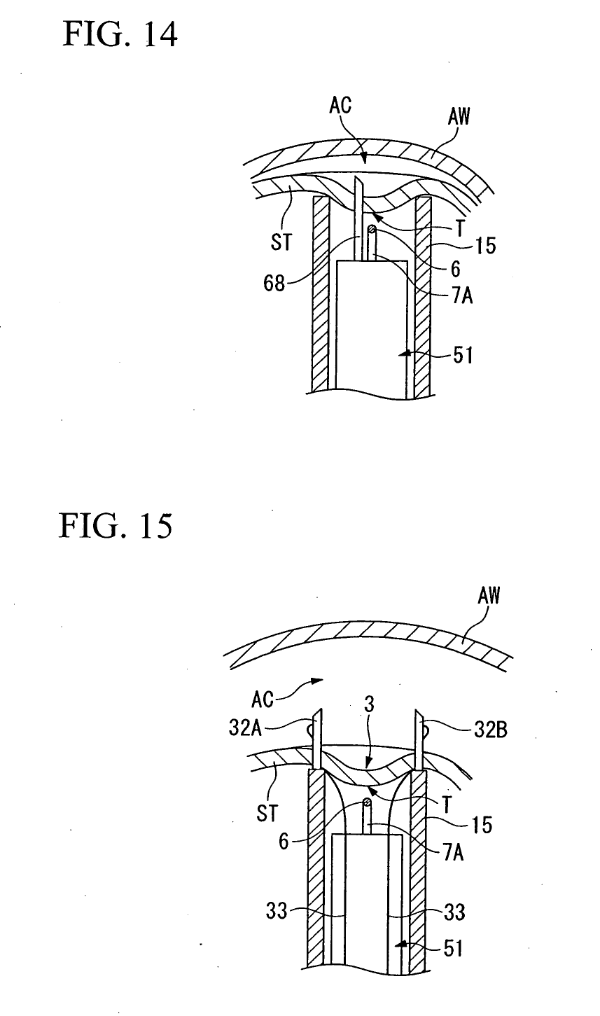

Next, the process proceeds to an insufflation step (S42). First, the insufflation needle 68, which is connected to the air and water feeding apparatus 62, is inserted into the channel 60 of the endoscope 2. Then the distal end of the insufflation needle 68 is projected into the distal end portion 15, and as shown in FIG. 14, inserted farther up to the abdominal cavity AC by puncturing the stomach wall that has suction applied. Thereby, while the space between the stomach wall having suction applied and the abdominal wall AW is established, the stomach wall is punctured by the insufflation needle 68, and thereby it is possible to puncture reliably only the stomach wall. Next, air is passed into the abdominal cavity AC via the insufflation needle 68 and the abdominal cavity AC is inflated so that the stomach ST and the abdominal wall AW are separated.

Preferably, the length of this insufflation needle 68 is about 12 mm, and more preferably, the distal end can be bent so as to be able to penetrate the center of the stomach wall that has the suction applied. In the case of a bent injection needle, there is a bend at the distal end, and a bent wire (not illustrated) is installed that passes through the inside of the bend in the radial direction from the distal end toward the proximal side. Here, the channel 60 of the endoscope 2 is disposed between the 6 o'clock and the 8 o'clock direction of the endoscope insertion portion 51, and thus when the anterior wall of the stomach ST, the preferable incision position, is cut, the approach is from an upward angle. Therefore, the bent wire faces the center because the bend follows the condition of the bending of the insertion portion 5 of the overtube 1, and thus by pulling the bent wire toward the proximal side, it is possible to puncture the center of the stomach wall reliably. In addition, when using a normal injection needle without a bending capacity, the bending control of the endoscope 2 is carried out in the overtube 1, and the injection needle thereby faces the middle. Note that during insufflation, the inside of the abdominal cavity AC may be maintained at an appropriate pressure by a feed gas pressure monitor and automatic control.

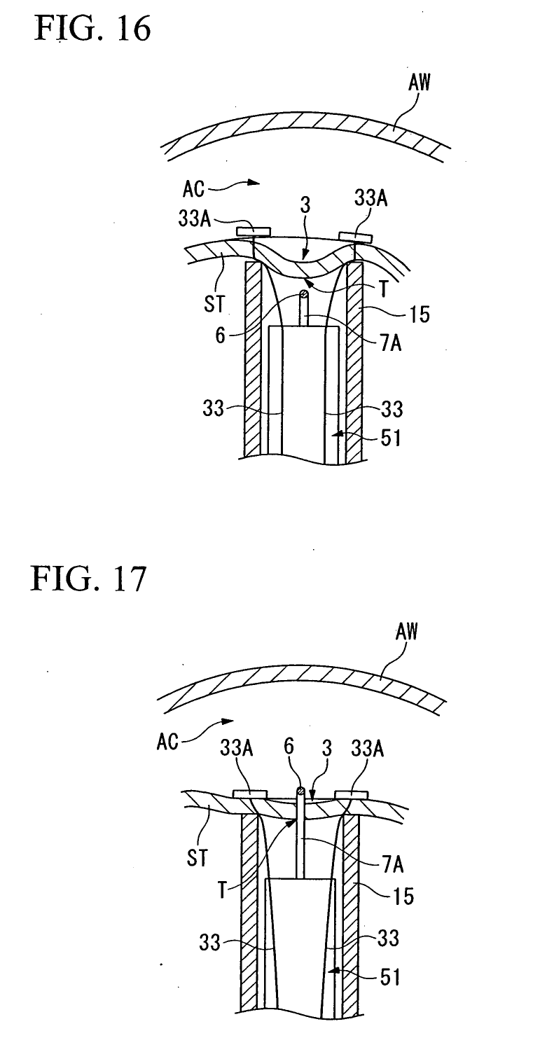

Then the process proceeds to a retention step (S43). Here, first the sheath grasping portion 40 is grasped and the needle controlling handle 41 is advanced toward the sheath grasping portion 40. Thereby, as shown in FIG. 15, the puncture needles 32A and 32B puncture the stomach wall by respectively projecting from the first outer groove 30 and the second outer groove 31 of the distal end portion 15 toward the distal end side. Specifically, the puncture needles 32A and 32B, which hold the anchors 33A, penetrate both sides of the tissue such that the targeted incision site T is situated therebetween from the anterior side (the inside of the stomach wall) to reach the posterior side (the outside of the stomach wall). From this state, the pusher connecting portion 43 is advanced toward the needle controlling handle 41 to move the pushers 35 in the distal end direction of the puncture needles 32A and 32B. At this time, as shown in FIG. 16, the anchors 33A of the thread members 33 are pushed by the pusher 35 and delivered from the hollow portion of the puncture needles 32A and 32B into the abdominal cavity AC. Thereby, the two anchors 33A are each retained on the posterior side of the tissue in proximity to the targeted incision site T on both sides thereof such that the targeted incision site T is situated therebetween. Here, because the AC is insufflated and a space around the stomach wall is established, it is possible to puncture only the stomach wall.

After the anchors 33A of the thread members 33 have been delivered, the pusher connection portion 43 is retracted with respect to the needle controlling handle 41, and furthermore, the needle controlling handle 41 is retracted with respect to the sheath grasping portion 40 and thereby the puncture needles 32A and 32B are again accommodated in the first outer groove 30 and the second outer groove 31. At this time, the anchors 33A of the thread members 33 form a T-shape due to the bending of the thread members 33. Subsequently, the sheath grasping portion 40 is grasped, the puncture needles 32A and 32B are removed from the distal end portion 15 by pulling the distal side, and then removed from the overtube 1. Thereby, the bending characteristics of the bending portion 13 are ensured.

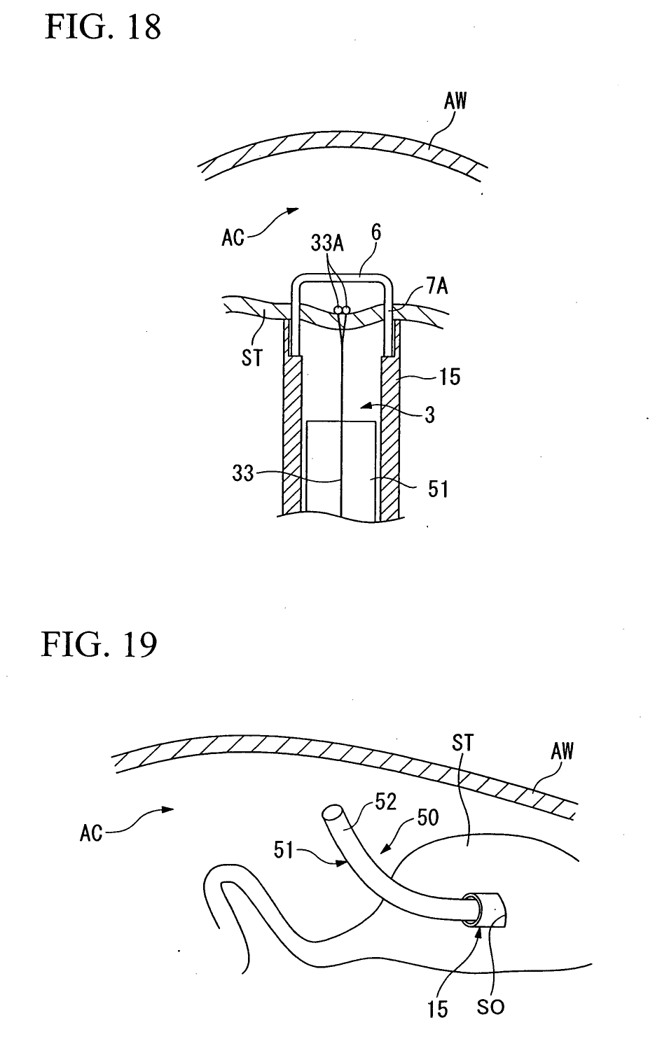

Then the process proceeds to an incision step (S50). First, it is confirmed that the connecting terminal 26B of the power cord 28 is connected to the connecting terminal 26A of the electrode controlling portion 8. Then while supplying a high frequency power from a high frequency power source 27, the controlling handle 21 is advanced with respect to the controlling body portion 20, and the cutting electrode 6 is projected from the distal end portion 15 to abut the stomach wall. Simultaneously, the proximal end side of the two thread members 33, which are exposed from the proximal end side opening of the overtube, are pulled in a distal direction. Thereby, the tissue that is present on both sides of the targeted incision site T can be pulled to the distal end side of the overtube 1, that is, to the cutting electrode 6 side, by each of the anchors 33A that are attached to the distal end of both thread members 33. Then the cutting electrode 6 is charged via the electrode controlling wires 7A and 7B, and thereby, as shown in FIG. 17 and FIG. 18, the cutting electrode 6 cuts the stomach wall, and an opening SO is formed in the stomach wall. Note that during this step also, by continuing to apply the suction to the stomach wall while simultaneously pulling the proximal end sides of the thread members 33 toward the proximal side, during the incision it is possible to prevent more completely the misalignment of the targeted incision site T. Here, while the two thread members 33 are pulled in a proximal direction and the tissue that is present at both sides of the targeted incision site T is pulled toward the distal end side of the overtube 1, the cutting electrode 6 may be pushed in the direction opposite to the direction in which the tissue is drawn (the forward direction of the overtube 1) to cut the tissue. Specifically, when the cutting electrode 6 is disposed so as to move freely (advance and retract freely) with respect to the overtube 1, it is possible to abut the cutting electrode 6 against the tissue and thereby cut the tissue while the tissue is being pulled.

Next, the process proceeds to a removal step (S60). Here, in order to remove the cutting electrode 6 from the insertion portion 5, the fastening screw 24 on the control body portion 20 of the electrode controlling portion 8 is loosened. At this time, the electrode controlling wires 7A and 7B are separated from the connecting plate 23, and the electrode controlling wires 7A and 7B are detached. Then, for example, by grasping and then pulling the end portion of the electrode controlling wire 7A toward the distal side, the electrode controlling wire 7A is moved through the inside of the first lumen 3 to the proximal end side, and the electrode controlling wire 7B is moved through the inside of the first lumen 3 to the distal end side. Finally, the electrode controlling wire 7B also moves to the proximal end side by passing through the distal end opening of the first lumen 3. In this manner, the cutting electrode 6 is extracted along with the electrode controlling wires 7A and 7B.

Next, the process proceeds to an introduction step (S70). Specifically, as shown in FIG. 19, the endoscope insertion portion 51 of the endoscope 2, which is the treatment device, is introduced into the abdominal cavity AC by passing through the opening SO. At this time, when it is necessary to restrict the relative motion between the insertion portion 5 and the endoscope insertion portion 51, an endoscope lock button 47 is pressed to abut the endoscope insertion portion 51, and due to the friction therebetween, the movement of the endoscope insertion portion 51 is prevented. Because the endoscope lock button 47 is provided, it is possible to inhibit the movement of the endoscope 2 relative to the overtube 1 by operating the endoscope lock button 47, and it is possible to insert both the overtube 1 and the endoscope 2 into the body simultaneously. In addition, because the operation of inserting the endoscope 2 is carried out while holding the distal handle 44 of the overtube 1, one hand of the operator can support the insertion portion 5 of the overtube 1 while the other hand can carry out the operation of grasping the distal handle 44, and thereby the operability is further improved.

Next, using the endoscope 2 that has been inserted into the abdominal cavity AC, a treatment step (S80) is executed in which various manipulations (medical procedures) including the observation of the organs in the abdominal cavity AC, incision, aspiration of cells, suturing, or the like are carried out.

After the treatment has been carried out, the overtube 1 and the endoscope 2 are extracted from the opening SO of the stomach.

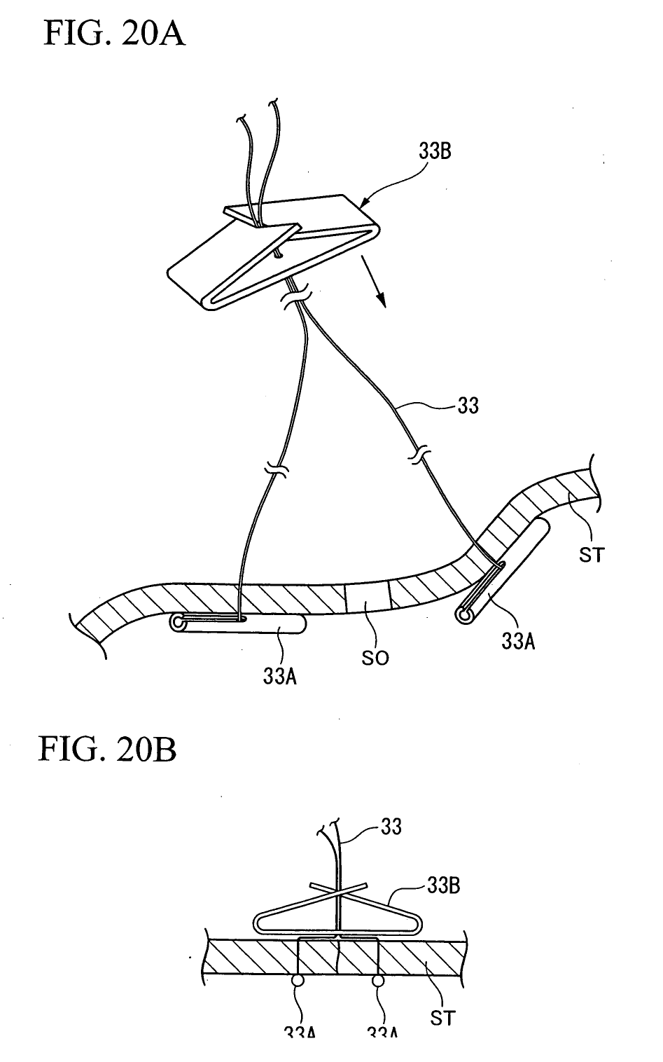

In the suturing step (S90), as shown in FIG. 20, the stopper 33B is engaged to the two thread members 33 that are exposed at the proximal end side of the overtube 1.

Next, the stopper 33B will be explained. The stopper 33B has a hole through which the thread members 33 pass at the center of an elongated plate member in the longitudinal direction. Both end portions of the stopper 33B in the longitudinal direction can be bent back at an angle to hold the thread members 33. Both end portions of the stopper 33B in the longitudinal direction are cut into triangular shaped notches. Both end portions of the stopper 33B can be bent back at an angle such that the notches intersect, and the thread members 33 can be held there.

In addition, as shown in FIG. 20, the two thread members 33 pass through the hole at the center of the plate member, and at the same time, the stopper 33B engages the two thread members 33 such that the proximal end sides of the thread members 33 that have been passed through the hole are held by the intersecting portions of both ends of the plate member that have been bent back at an angle. In this state, the thread members 33 cannot fall out from between the proximal portions. In addition, when the proximal end sides of the thread members 33 are pulled in the direction of separation from the stopper 33B, both end portions of the stopper 33B open slightly. Thereby, the stopper 33B permits movement of the thread members 33 in this direction. In contrast, when the thread members 33 are pulled toward the distal end side with respect to the stopper 33B, the thread members 33 move in the direction indicated by the arrow in FIG. 20A. However, at this time, because both end portions of the stopper 33B close to clamp the thread members 33, the thread members 33 cannot move. That is, as shown in FIG. 20, when the stopper 33B engages the thread members 33, the stopper 33B moves toward the distal end sides of the thread members 33, but does not move toward the proximal end side.

Then, the stopper 33B, which has been engaged in this manner, is moved up to the distal ends of the thread members 33. Here, a method of moving the stopper 33B up to the distal end positions of the thread members 33 may include, for example, covering the tube farther on the proximal end side than the portion engaged to the stopper 33B of the two thread members 33 and feeding the distal end of this tube toward the distal end sides of the thread members 33. Thereby, the stopper, which has been engaged to the distal end of the tube, moves integrally along with the tube.

As shown in FIG. 20B, by moving the stopper 33B up to the distal end sides of the thread members 33 in this manner, it is possible to bring the anchors 33B that are attached to the distal ends of the thread members 33 into proximity to each other, and thereby, it is possible to suture the opening SO. Note that the method for suturing the tissue by using the two thread members 33 is not limited to a method in which such a stopper is used. A knot may be formed by tying together the sections of the two thread members 33 that extend from the proximal end side of the overtube 1, feeding this knot into the distal end side of the overtube 1 by using a sheath-shaped member, and fastening the knots such that the knots do not shrink while the opening SO is sutured, thereby to suture the opening SO.

In the case that the suture is insufficient, depending on necessity, the suture may be supplemented by introducing other conventionally well-known devices.

After the suturing has been completed, the proximal end side portions of the thread members 33 near the engagement position of the stopper 33B are cut by a cutting instrument introduced from a channel of the endoscope 2, the proximal end sides of the thread members that have been cut are pulled out from the patient, and at the same time, the overtube 1 and the endoscope 2 are removed from the patient. Then, the pressure applied to the abdominal cavity AC is released and the manipulation is ended.

According to such a medical procedure, the distal end sides of the thread members 33 are fastened by the anchors 33A to the tissue in proximity to the targeted incision site T, and by pulling the proximal end sides of the thread members 33 during the incision while pressing the tissue in proximity to the targeted incision site T against the cutting electrode 6, which is the cutting instrument, the incision can be made. As a result, while making the incision, the cutting instrument is not displaced from the targeted incision site T, and it is possible to cut the targeted incision site more precisely. At the same time, it is possible to cut such that the cutting instrument reaches the posterior surface of the tissue reliably. In this connection, while making the incision, no tension is applied to the tissue that is to be cut, and if the cutting instrument is simply pressed against the tissue that is to be cut, for example, the tip of the cutting instrument frequently becomes displaced from the targeted incision site T when the surface of the tissue is very slippery or when the site that is to be cut is harder than another site. In the medical procedure of this embodiment, because the tissue in proximity to the targeted incision site T is cut while applying tension using the thread members 33, the tip of the cutting instrument becomes displaced from the targeted incision site T with difficulty.

In addition, because cutting is carried out by applying tension using the thread members 33 only to the tissue that is to be cut, it is possible to form a gap between the stomach wall, which is the tissue that is to be cut, and, for example, the abdominal wall in proximity thereto, and thereby it is possible to cut more easily only the necessary tissue.

In addition, in the medical procedure in this embodiment, two thread members 33 having the anchors 33A that are attached to the distal end thereof are used, the distal end sides of these thread members 33 are retained by the anchors 33A on both sides of the targeted incision site T so as to situate the targeted incision site T therebetween, and while pulling the proximal end sides of both of these thread members 33, the cutting electrode 6, which is the cutting instrument, is pressed against and cuts the targeted incision site T. Thereby, the displacement of the cutting instrument from the targeted incision site T is restricted on both sides, and it is possible to prevent more completely the displacement of the tip of the cutting instrument from the targeted incision site T.

In addition, because the thread members 33 are fastened in proximity to the tissue that is to be cut and the T-shaped anchors 33A that are attached to the distal ends of the thread members 33 are used, it is possible to fasten the distal ends of the thread members 33 to the tissue easily.

In addition, the hollow puncture needles 32A and 32B are used when the anchors 33A are fastened to the posterior side of the tissue. The anchors 33A are held beforehand in the hollow portion of these puncture needles 32A and 32B, and these puncture needles 32A and 32B penetrate so as to enter from the anterior side and reach the posterior side of the tissue that is to be cut. Subsequently, the anchors 33A are delivered from the hollow portion of these puncture needles. Thereby, the anchors 33A are disposed at the desired location. In addition, by using the hollow puncture needles 32A and 32B in this manner, it is possible to retain the anchors 33A at the desired location on the posterior side of the tissue extremely easily.

In addition, after the prescribed medical procedure in the abdominal cavity AC, by moving the stopper 33B engaged on the proximal end sides of the thread members 33 to the distal end side, it is possible to use the anchors 33A and the thread members 33 that are used while making the incision to suture the opening SO portion that has been cut, and thereby, it is possible to carry out the suturing operation more easily.

Furthermore, because a cutting electrode 6 is used that is disposed at the distal end side of the insertion portion 5 so as to traverse the distal end side of the lumen 3, it is possible to cut the stomach wall without preparing special instruments to be used for cutting when inserting the insertion member 5 into the stomach ST. In this situation, because the tissue is cut only by a length equivalent to the length of the cutting electrode 6 that traverses the lumen 3, it is possible to pass the overtube 1 through with only a light force, and it is possible to suppress suitably the leaks at the outer circumference of the overtube 1. In addition, because the electrode controlling wires 7A and 7B can be removed by the electrode controlling portion 8, it is possible to remove the cutting electrode 6 from the insertion portion 5 along with the electrode controlling wires 7A and 7B. Therefore, when the endoscope 2 projects from the lumen 3, the cutting electrode 6 does not become a hindrance, and when the endoscope 2 is passed through the lumen 3, it is possible to advance the endoscope 2 into the abdominal cavity AC beyond the tissue that has been cut.

Note that in the embodiment described above, the hollow puncture needles 32A and 32B that are used to retain the anchors are disposed on the outer circumferential surface side of the distal end portion 15 of the overtube 1, but this is not limiting. As shown in FIG. 21, the hollow puncture needles 32A and 32B for retaining the anchors may be disposed inside the distal end portion 15 of the overtube 1 (more specifically, the inner circumferential surface that defines the lumen 3). In this case, when the endoscope is disposed inside the overtube 1, due to this endoscope, it is possible to observe the condition of the tissue that is penetrated by the puncture needles 32A and 32B, and thus, it is possible to retain the anchors at the desired locations more reliably.

### Second Embodiment

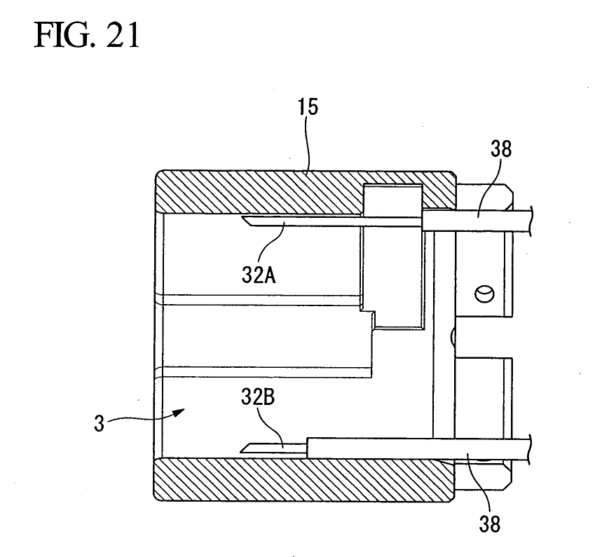

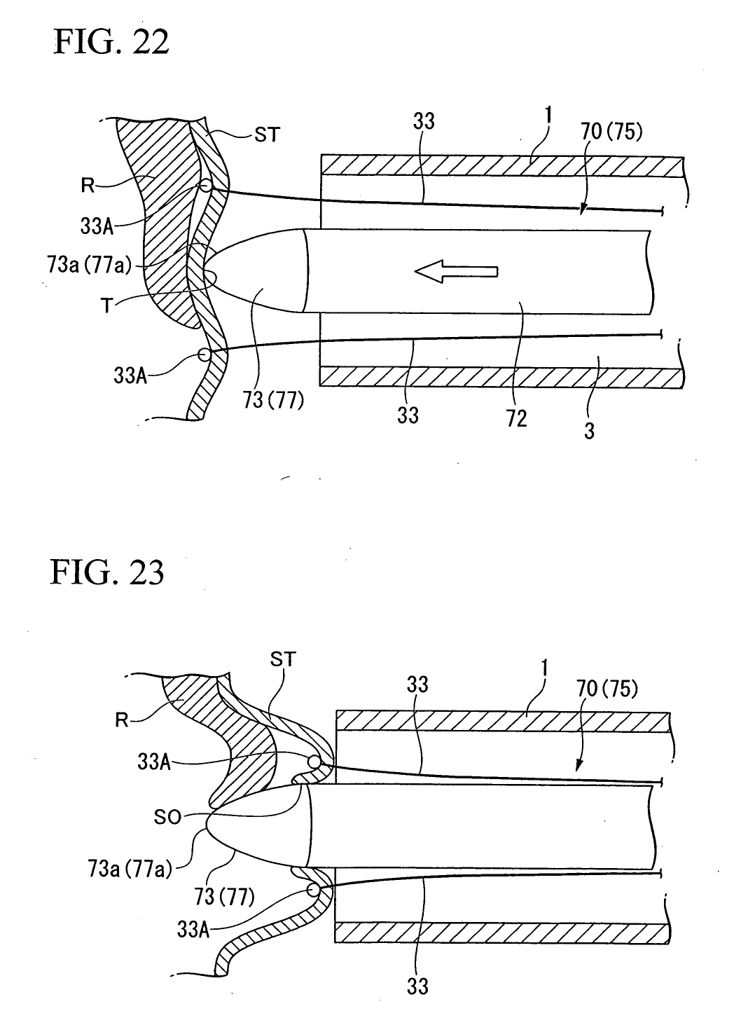

A second embodiment of the present invention will be explained with reference to FIG. 22 to FIG. 28.

The second embodiment differs from the first embodiment described above on the point that instead of using a high frequency, a knife shaped cutting instrument having a tip that can cut tissue on the distal end thereof is used.

Note that in the second embodiment, the cutting step using the knife shaped cutting instrument will be explained, but the other steps, specifically, the insertion step, the inflation step, the guiding step, the suction application step, the insufflation step, the retention step, the treatment step, and the suturing step, are identical to those of the first embodiment described above, and therefore the explanations thereof have been omitted here. This point is also identical with respect to the third and fourth embodiments.

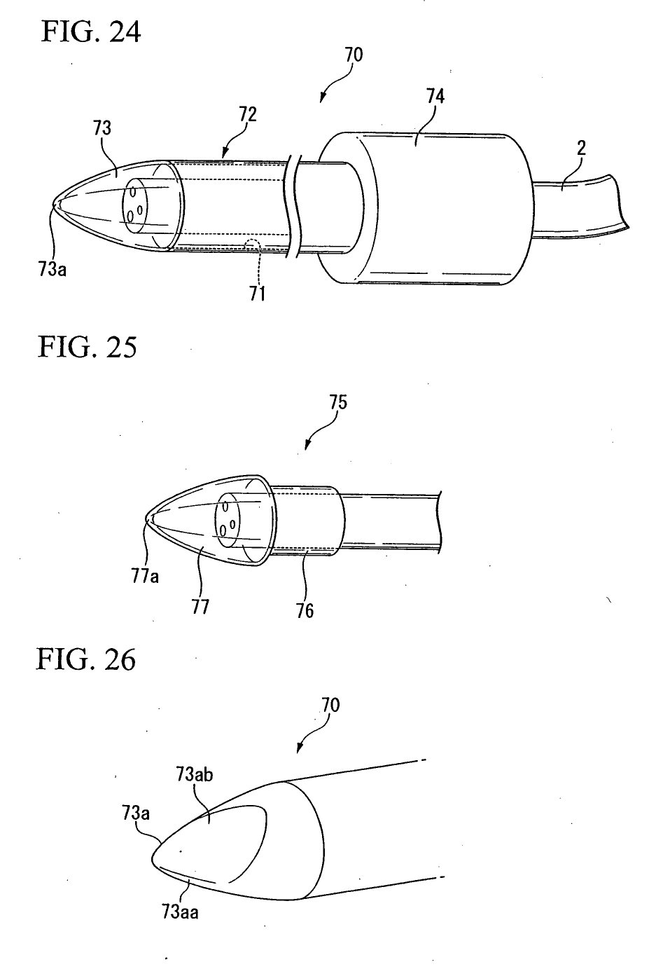

FIG. 22 and FIG. 23 are drawings for explaining the state in which, for example, the stomach wall ST is cut by using a knife shaped cutting instrument 70. As shown in FIG. 24, for example, it is possible to use what is called an overtube type cutting instrument as a cutting instrument 70. The overtube-type cutting instrument 70 has a tube shape and is provided with a lumen 71 through which a device such as an endoscope 2 can be passed.

The cutting instrument 70 shown in FIG. 24 is constructed by a tube portion 72, a cutting instrument body 73, and a grip portion 74. The tube portion 72 has an outer diameter that is set smaller than the inner diameter of the overtube 1 so as to be able to be pass through the overtube 1 and has a lumen 71 formed therein. The cutting instrument body 73 is attached to the distal end of the tube portion 72. The grip portion 74 is provided on the proximal end side of the tube portion 72, has a diameter that is larger than the outer diameter of the tube portion 72, and opens in the proximal end side. The cutting instrument body 73 is made from a transparent material such as glass or an acrylic resin, and on the distal end, a knife tip 73 is formed so as to be able to cut tissue. Note that, depending on necessity, a fastening device that fastens the endoscope 2 that passes through the inner lumen 71 may be provided on the cutting instrument 70 shown in FIG. 24, and thereby, the cutting instrument 70 and the inner endoscope 2 may be integrally constructed and inserted into the overtube 1.

The cutting instrument, as shown in FIG. 24 is not limited to what is called an overtube type. As shown in FIG. 25, what is called an attachment type cutting instrument 75 that is releasably attached to the distal end of the endoscope 2 may be used.

The attachment-type cutting instrument 75 is structured by a fitting portion 76 that fits over the outside of the distal end of the endoscope 2, and a cutting instrument body 77 that is attached to the distal end of the fitting portion 76 and that has a tip 77a. It is not necessary for the entire cutting instrument 75 to be transparent, but preferably at least a portion of the cutting instrument body 77 is made of a transparent material so that the forward visibility of the endoscope 2 to which the cutting instrument 75 is attached can be ensured. A method that can be considered for attaching the cutting instrument 75 to the endoscope 2 is forming the fitting portion 76 of the cutting instrument 75 from an elastic material and fitting the fitting portion 76 onto the distal end of the endoscope 2 by using this elasticity.

The shape of the tips 73a and 77a of the cutting instrument body of the cutting instruments 70 and 75 is not limited to the substantially conical shapes shown in FIG. 24 and 25, but in addition, the various shapes shown in FIG. 26 to FIG. 28 can be considered. However, while FIG. 26 to FIG. 28 show examples of the tip shape of what is called an overtube-type cutting instrument, of course this is not limiting. These shapes can similarly also be applied to what is called an attachment-type cutting instrument.

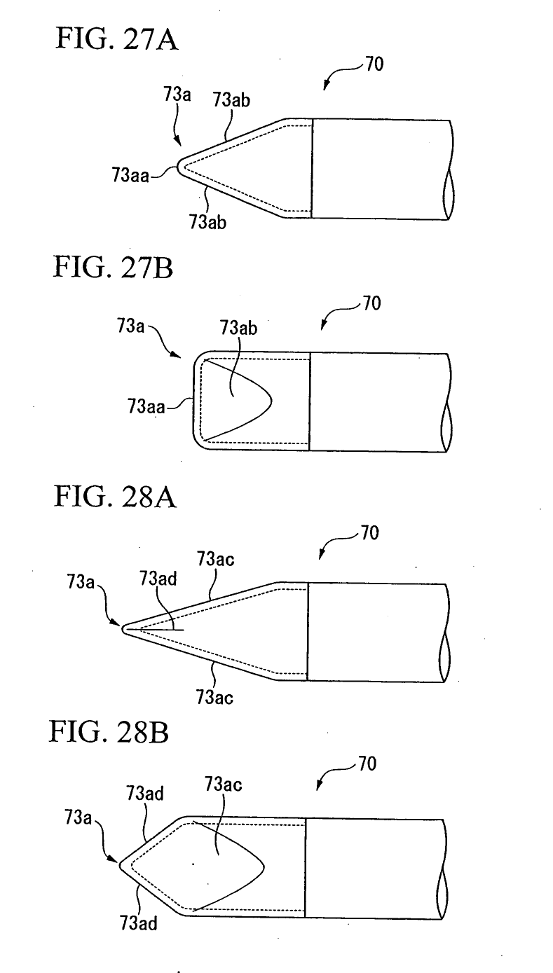

FIG. 26 and FIG. 27 show what is called a knife edge shaped cutting instrument, which has inclined surfaces 73ab respectively on the right and left, the width of these left and right inclined surfaces 73ab gradually narrow toward the distal end, and a flat portion 73aa is formed on the distal end. In addition, the cutting instrument shown in FIG. 28 has a shape in which there are left and right inclined surfaces 73ac, the width of the left and right inclined surfaces 72ac gradually narrows toward the distal end, and at the same time, when viewing these inclined surfaces 73ac frontally, the distal end tapers to a point similar to an arrow such that the vertical ridge line 73ad is approximately 90 degrees. Each of the tips 73a shown in FIG. 26 to FIG. 28 forms what is called a dull tip, where the radius of curvature of the distal end thereof is approximately 0.05 mm to 0.5 mm, and this is not particularly sharp. Therefore, this dull tip cannot cut the tissue simply by being pressed against the tissue using a weak force. Unless the dull tip is pressed by a relatively strong force, the tissue cannot be cut.

In the explanation of the medical procedure using this cutting instrument, as described above, by using, for example, the lumen 3 of the overtube 1, the anchors 33A that are attached to the distal ends of the thread members 33 are each retained on the posterior side of the tissue (for example, the stomach wall) that is to be cut on both sides of the targeted incision site T (FIG. 22).

In addition, the endoscope 2 is temporarily removed from the overtube 1, and the overtube type cutting instrument 70 described above is fit onto the outer circumference of the removed endoscope 2. Alternatively, the attachment type cutting instrument 75 described above may be attached to the distal end of the removed endoscope 2.

Then the cutting instrument 70 or the cutting instrument 75 attached in this manner is inserted into the lumen 3 of the overtube 1, and the cutting instrument body 73 (77) on the distal end reaches the vicinity of the targeted incision site T. In this state, the cutting instrument 73 (77) is then pressed farther toward the distal end side. Simultaneously, the proximal end sides of the two thread members 33 that are exposed from the proximal end side opening of the overtube 1 are pulled in the distal direction. Thereby, as shown in FIG. 22, it is possible to pull the tissue that is present on both sides of the targeted incision site T toward the tip 73a of the cutting instrument body 73 (77) by using each of the anchors 33A that are attached to the distal ends of both thread members 33. As a result, as shown in FIG. 23, the stomach wall ST is cut by the tip 73a (77a) of the cutting instrument body 73 (77), and it is thereby possible to form an opening SO in the stomach wall ST.

Here, the tip 73a (77a) of the cutting instrument body 73 (77) may contact other organs such as the liver R and the abdominal wall which are adjacent to the stomach wall ST. However, because the tip 73a (77a) of the cutting instrument body has a dull shape rather than a sharp one and because tension is not applied to the tissue of the liver R or the like, which are not the objects of the incision by the thread members 33, the tip 73a (77a) of the cutting instrument body does not cut the tissue of the liver R and the abdominal wall or the like, and does not enter into these tissues.

After the incision has been completed, the cutting instrument 70 (77) is again removed from the endoscope 2 and the overtube 1, and the cutting instrument 70 (75) is separated from this removed endoscope 2. Then the endoscope 2, without the cutting instrument attached, is again inserted into the lumen 3 of the overtube 1, and the desired manipulation is carried out in the abdominal cavity AC.

### Third Embodiment

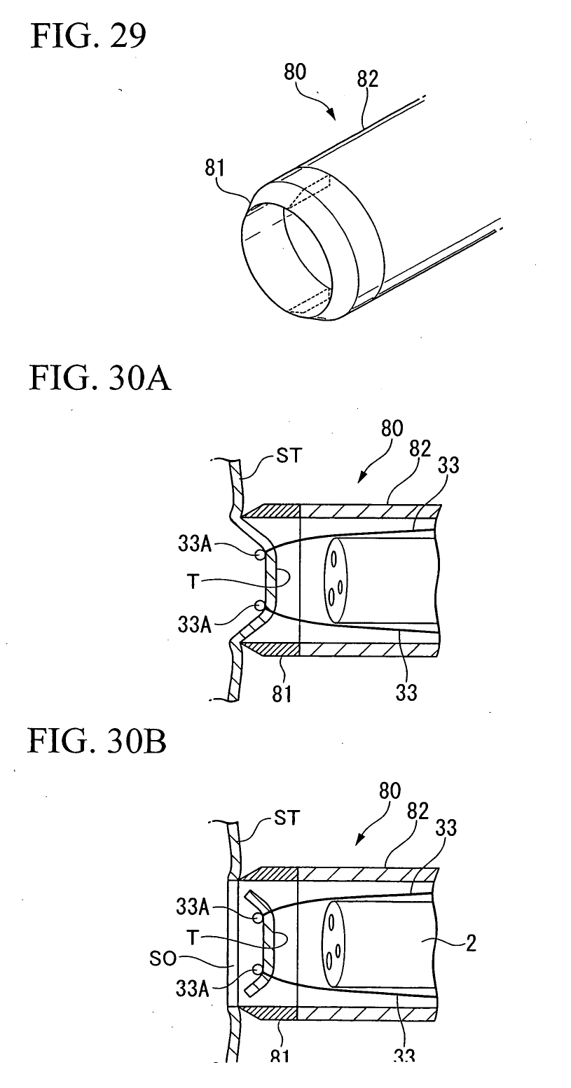

A third embodiment of the present invention will be explained with reference to FIG. 29 to FIG. 31.

The third embodiment differs from the first embodiment described above on the point that instead of using a high frequency, on the distal ends of the overtubes 80 and 90, tip portions 81 and 91, which are integrally mounted on the overtubes 80 and 90 and can cut tissue, are used as cutting instruments.

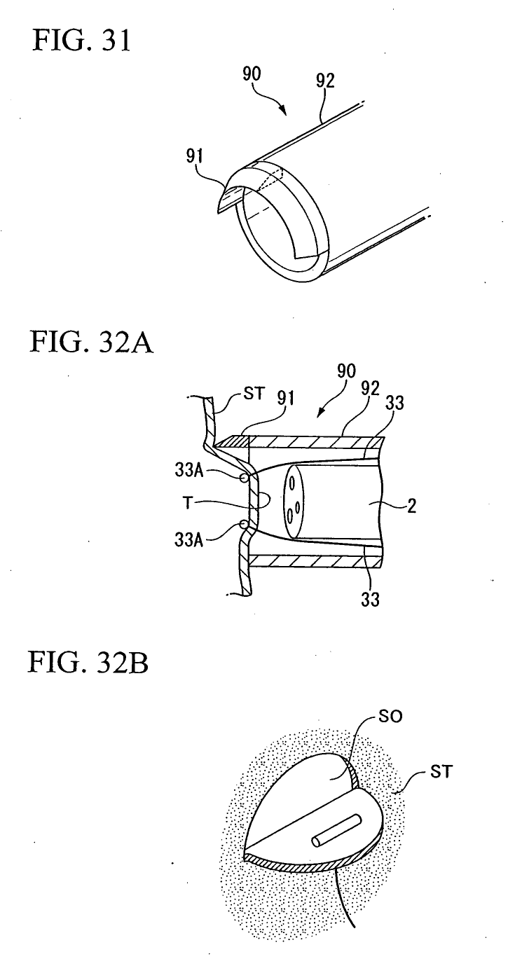

Examples of the tip are, as shown in FIG. 29, the tip portion 81 that is attached along the entire circumference of the distal end of the overtube 80, and as shown in FIG. 31, the tip portion 91 that is only installed on a portion, for example, the upper half, of the distal end of the overtube 90.

The example shown in FIG. 29 is one in which a tip portion 81 made of a metal such as stainless steel is attached to the distal end of the overtube body 82, which is made of a flexible material or a material having a certain degree of rigidity. The tip of this tip portion 81 is preferably what is called a dull tip, in which the radius of curvature of the distal end thereof is comparatively large and not necessarily sharp. This is to prevent damaging tissue that is not the object of cutting.

A medical procedure using this cutting instrument will now be explained. The anchors 33A that are attached to the distal ends of the thread members 33 are retained by establishing in advance a predetermined gap in proximity to the targeted incision site T. The overtube 80 is advanced, and the tip portion 81 that is attached to the distal end thereof reaches the vicinity of the targeted incision site T. In this state, when the overtube 80 is pushed slightly toward the distal end side, simultaneously, the proximal end side of the two thread members 33 that are exposed from the proximal end side opening of the overtube 80 are pulled toward the distal side. Thereby, as shown in FIG. 30A, the tissue that is present at the targeted incision site T can be pulled to the tip portion 81 of the distal end of the overtube by each of the anchors 33A that are attached to the distal ends of both thread members 33. Then, by pulling the thread members 33 with a stronger force, as shown in FIG. 30B, the stomach wall ST is cut by the tip portion 81, and it is thereby possible to form a circular opening SO in the stomach wall ST.

After the incision has been completed, using this opening SO portion, the desired medical procedure is carried out by introducing the distal end of the overtube 80 and the insertion portion of the endoscope 2, which is inside the overtube, into the abdominal cavity.

In contrast, as shown in FIG. 31, the tip portion 91, which is made, for example, of metal and formed only on the upper half of the overtube 92, is mounted on the distal end of the overtube body 92, which is made of a flexible material or a material having a certain degree of rigidity. The tip of this tip portion 91 is preferably what is called a dull tip, in which the radius of curvature of the distal end thereof is comparatively large and not necessarily sharp. This is to prevent more completely damaging the tissue that is not the object of cutting.

In the medical procedure using this cutting instrument, the anchors 33A that are attached to the distal ends of the thread members 33 are retained on the posterior side of the tissue (for example, the stomach wall) that is to be cut and a predetermined gap in proximity to the targeted incision site T is established in advance. Then the overtube 90 that has reached the vicinity of the targeted incision site T is pressed slightly toward the distal end side, and simultaneously, the two thread members 33 that are exposed from the proximal end side opening of the overtube 90 are pulled toward the distal side. Thereby, as shown in FIG. 32A, the tissue that is present at the targeted incision site T can be pulled to the tip portion 91 of the distal end of the overtube by the anchors 33A that are attached to the distal ends of both thread members 33. By pulling the thread members 33 with a stronger force, as shown in FIG. 32B, the stomach wall ST is cut by the tip portion 91, and it is thereby possible to form a semi-circular opening SO in the stomach wall ST.

Note that an overtube into which the overtube 80 shown in FIG. 28 to FIG. 31 is inserted, for example, an overtube having a structure in which the cutting electrode is eliminated from the overtube shown in the first embodiment, may also be used as a device into which the cutting instrument (overtube 80) is inserted. In this case, it is possible to make the tip portion 81 sharp.

### Fourth Embodiment

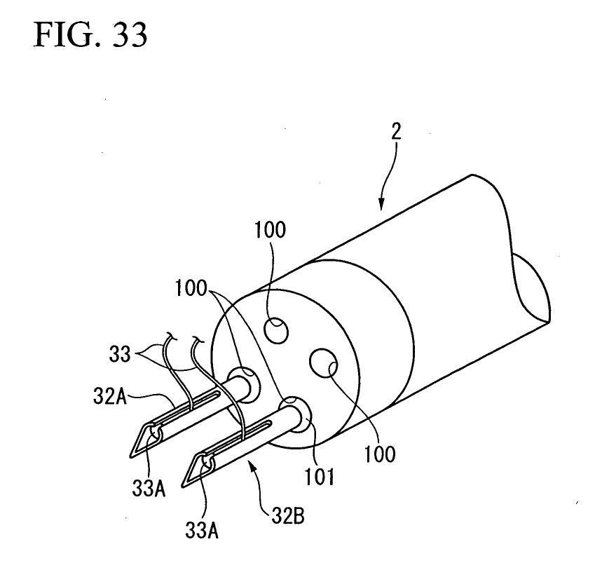

A fourth embodiment of the present invention will be explained with reference to FIG. 33 to FIG. 36.

The fourth embodiment differs from the first embodiment described above on the point that the anchors of the thread members 33 are retained on the posterior side of the tissue by using a channel of the endoscope 2 instead of an overtube, and the point that the proximal end sides of the thread members 33 are pulled outside the body of the patient directly, without using the lumen of an overtube.

Specifically, in this embodiment, the outside sheath 101 having hollow puncture needles 32A and 32B in the distal end thereof is inserted in advance into a channel 100 of the endoscope 2. Then each of the anchors 33A is set by being held in the hollow puncture needles 32A and 32B, and at the same time, the thread members 33 that extend from the anchors 33A are passed through the outside sheath 101 or through another channel 100 of the endoscope.

The endoscope 2 in which the outer sheath 101 or the like has been set in advance is inserted into the stomach (a luminal organ) through the mouth of a patient. Then the insertion portion of the endoscope 2 is brought into proximity to the targeted incision site. After the targeted incision site has been confirmed, the needle controlling portion is operated (not illustrated), the puncture needle 32A is projected from the distal end of the endoscope 2, and the tissue in proximity to the targeted incision site is punctured. In this state, a pusher control portion (not illustrated) is operated, and the anchors 33A are delivered by the pushers from the hollow portion of the puncture needle 32A and retained on the posterior side of the tissue. In contrast, a similar operation is carried out with the puncture needle 32B, which has been set in the other channel, and the anchor 33A is delivered to and retained on the posterior side of the tissue on the other side such that the targeted incision site is situated therebetween.

Next, the endoscope that has delivered the anchors 33A is temporarily removed through the mouth of the patient, and the overtube type cutting instrument 70 explained in the second embodiment is fit on the outer circumference of this extracted endoscope 2 or the attachment type cutting instrument 75 is attached to the distal end of the endoscope 2.

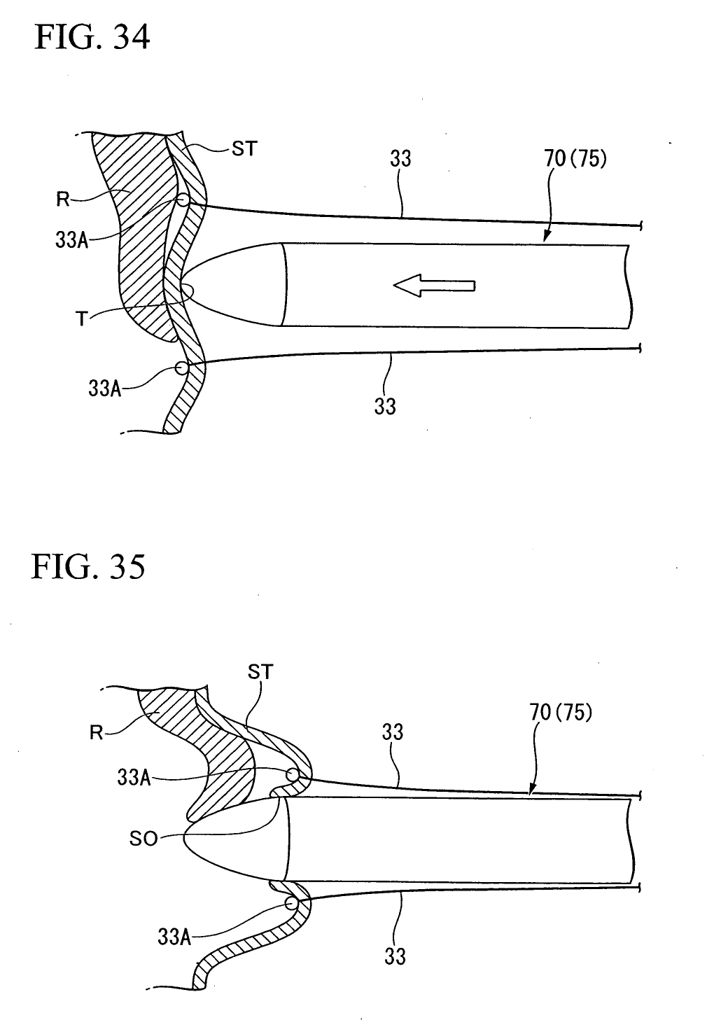

In addition, this cutting instrument 70 or the cutting instrument 75 is again inserted integrally with the endoscope 2 into the stomach (a luminal organ) ST through the mouth of the patient. Then the cutting instrument 70 (75) at the distal end of the endoscope reaches the vicinity of the targeted incision site T, and in this state, the cutting instrument 70 (75) is pushed farther in the distal end direction. Simultaneously, the proximal end sides of the two thread members 33, which are exposed from the proximal end side opening of the overtube 1, are pulled distally. Thereby, as shown in FIG. 34, the tissue that is present on both sides of the targeted incision site T can be pulled to the tip of the cutting instrument body 73 (77) by each of the anchors 33A that are attached to the distal ends of both thread members 33. Then by pulling the thread members 33 with a stronger force, as shown in FIG. 35, the stomach wall ST is cut by the tip of the cutting instrument body 73 (77), and thereby it is possible to form an opening SO in the stomach wall ST.

Note that the technical scope of the present invention is not limited by the embodiments described above, and various modifications may be added within scope thereof that do not depart from the spirit of the present invention.

For example, in the embodiments described above, a flexible endoscope is used as the observing apparatus, but this is not limiting. For example, in the embodiments described above, carrying out the desired manipulations by introducing an endoscope having an observing apparatus into the abdominal cavity was described, but this is not limiting. For example, an observing instrument called a capsule endoscope may be retained in the abdominal cavity. The desired manipulation may be carried out by passing a device that does not have an observing apparatus through the insertion portion into the overtube while observing the manipulation using the capsule endoscope.

In addition, in the first embodiment, as shown in FIG. 4, the length of the cutting electrode 6 is set such that the cutting electrode 6 can be accommodated in the lumen 3 in a bent state, however this is not limiting. The length of the cutting electrode 6 may be set such that the cutting electrode 6 can be accommodated in the lumen 3 in a state that is not bent. The length of the cutting electrode (the length of the portion that traverses the lumen) may be appropriately set depending on the outer diameter of the overtube itself or the outer diameter of the device that is passed through the lumen. Thereby, the only the minimum opening needs to be formed. Therefore, when devices are introduced into the abdominal cavity by being passed through the opening in the luminal organ that has been formed by using a cutting electrode, it is possible to reduce to a minimum the gap formed between the device and the opening, and it is possible to ensure the air-tightness between the inside of the luminal organ and the abdominal cavity at a degree to which the pressure applied to the abdominal cavity AC can be maintained. In addition, the number of puncture needles is not limited to two. Four or six puncture needles may be disposed in a prescribed gap in the circumferential direction.

What is claimed is:

1. A medical procedure carried out via a natural opening, comprising:
fastening the distal end of thread members to a tissue in a body cavity in proximity to a targeted incision site via a first introduction member that is inserted into a body cavity through a natural opening of an examination subject;
inserting a second introduction member having a cutting instrument attached to the distal end thereof into a body cavity through a natural opening of an examination subject and disposing the cutting instrument in proximity to the targeted incision site; and
cutting the tissue by pressing the cutting instrument against the same while pulling the proximal end sides of the thread members.

2. A medical procedure carried out via a natural opening according to claim 1, further comprising:
fastening the distal ends of the thread members to the tissue in proximity to the targeted incision site by retaining the anchors attached to the distal ends of the thread members on the posterior side of the tissue in proximity to the targeted incision site.

3. A medical procedure carried out via a natural opening according to claim 2, further comprising:
retaining the anchors on the posterior side of the tissue in proximity to the targeted incision site by hollow puncture needles that have been passed through a lumen provided on the first introduction member entering so as to reach from the anterior side to the posterior side of the tissue and the anchors being retained after passing through the hollow portion of the puncture needles on the posterior side of the tissue.

4. A medical procedure carried out via a natural opening according to claim 3, further comprising:
providing two sets of a thread member and an anchor attached to the distal end sides thereof, and retaining each of the two anchors on both sides of the targeted incision cite so as to situate the targeted incision cite therebetween.

5. A medical procedure carried out via a natural opening according to claim 4, further comprising:
using the anchors and the thread members to suture the opening portion that has been cut.

6. A medical procedure carried out via a natural opening according to claim 5, wherein the cutting instrument uses a high frequency.

7. A medical procedure carried out via a natural opening according to claim 5, wherein the cutting instrument is one in which the radius of curvature of the tip is set within a range of 0.05 mm to 0.5 mm.
